(19) United States
(12) Patent Application Publication
(10) Pub. No.: US 2024/0197698 A1
(43) Pub. Date: Jun. 20, 2024

(54) TREATMENT OF ACUTE ANXIETY WITH AN ALPHA-7 NICOTINIC ACETYLCHOLINE RECEPTOR MODULATOR

(71) Applicant: Vanda Pharmaceuticals Inc., Washington, DC (US)

(72) Inventors: Mihael H. Polymeropoulos, Potomac, MD (US); Yunsheng He, Fairfax, VA (US); Christos Polymeropoulos, Potomac, MD (US)

(21) Appl. No.: 18/527,638

(22) Filed: Dec. 4, 2023

Related U.S. Application Data
(60) Provisional application No. 63/385,896, filed on Dec. 2, 2022.
(60) Provisional application No. 63/504,431, filed on May 25, 2023.

(51) Int. Cl. A61K 31/439; A61P 25/22

(52) U.S. Cl. CPC A61K 31/439; A61P 25/22

(57) ABSTRACT

Disclosed herein are methods of treatment or prevention of acute anxiety in an individual in need thereof. Such methods include administering to the individual an alpha 7 nicotinic acetylcholine receptor (α7 nAChR) modulator at a therapeutic dose to treat or prevent manifestations of the acute anxiety and symptoms thereof.

### CROSS REFERENCE TO RELATED APPLICATIONS

The present patent application claims priority to U.S. Provisional Patent Application No. 63/385,896, filed Dec. 2, 2022, and U.S. Provisional Patent Application No. 63/504,431, filed May 25, 2023, each of which is incorporated by reference as though fully set forth herein.

### BACKGROUND OF THE INVENTION

The invention relates to a method for the treatment of acute anxiety. More particularly, the invention relates to a method for the treatment of acute anxiety such as, e.g., performance anxiety, public speaking anxiety, procedural anxiety, acute phobia situations, and other acute anxieties with an alpha 7 nicotinic acetylcholine receptor (α7 nAChR) modulator, particularly an α7 nAChR modulator with agonist activity.

Acute anxiety manifests in individuals in varied settings, and due to varied causes. For example, acute performance anxiety may be triggered by an event such as a musical performance, or an oral or written test. Acute public speaking anxiety (PSA) may be triggered by a public speaking engagement. Acute procedural anxiety may be triggered by a medical or dental procedure. Acute anxiety may also be triggered by experiencing an acute phobia setting or activity such as, e.g., boarding, taking off, or landing in an airplane, experiencing a high height such as, e.g., mountain climbing or standing on an upper floor of a tall building, proximity to an anxiety-inducing animal such as, e.g., an arachnid or snake, and other anxiety-inducing settings and/or phobic exposures. Acute anxiety may also manifest as a result of social anxiety disorder (SAD), SAD-performance only (SAD-PO), public speaking anxiety (PSA) independent of SAD diagnosis, public speaking anxiety as a presenting symptom of social anxiety disorder (SAD), public speaking anxiety as a presenting symptom of SAD-performance only (SAD-PO).

Unlike generalized anxiety, which may affect an individual's ability to function in daily life, acute anxiety is typically associated with a triggering event. In the case of procedural anxiety, a medical or dental procedure may be the triggering event. Symptoms of acute anxiety may include, e.g., sweating, feeling of breathlessness, irritability, shaking/trembling, heart palpitations, pounding heart, or increasing heart rate, loss of appetite, inability to speak or think clearly, a sensation of detachment from reality, fear of fainting, and fear of losing control. These symptoms can be so overwhelming that it disrupts a person's ability to function before, during, or even after the procedure has ended. Treatment may be beneficial when anxiety builds to a level at which it causes distress to the sufferer and/or interferes with the individual's ability or willingness to start or complete a procedure, and to cope effectively after a procedure is completed. However, current pharmacological interventions include sedation with benzodiazepines, nitrous gas, and general anesthesia, each of which is limited in its utility, at least in part due to risks and an unfavorable balancing of costs and benefits. For example, general anesthesia is incompatible with certain triggers, such as performance, and in other instances may result in serious cognitive consequences which may linger for weeks afterward. In rare cases, individuals may never regain consciousness. Sedation may impact an individual's performance during the anxiety-inducing event, as well as afterward.

Performance anxiety, an excessive fear of speaking or performing in public situations, is a common cause of acute anxiety. Performance anxiety has been reported to affect as many as 80% of SAD patients, approximately 40% of whom experience severe performance anxiety. The most commonly feared situation reported in performance anxiety is public speaking anxiety, which has an estimated prevalence of up to 20% of the general population. Public speaking anxiety (PSA) is classified as a social anxiety disorder (SAD) in the American Psychiatric Association's Diagnostic and Statistical Manual of Mental Disorders, 5th edition (DSM-5) (2013). Sufferers of performance anxiety report debilitating anxiety that ranges from public speaking to test taking, writer's block, and musical performance. People with performance anxiety report that the condition can hinder or even threaten their professional career of choice. Similarly, musicians report that performance anxiety makes them perform at levels below their capabilities. Currently, there is no FDA-approved drug treatment available for performance anxiety, and general anti-anxiolytic drugs tend to be sedating which is not preferred in a performance situation.

Social anxiety disorder (SAD), including performance anxiety and PSA, is characterized by excessive fear, discomfort, and self-consciousness in a variety of social situations including, e.g., performing in front of others, and giving a speech. SAD is a chronic condition with a typical onset during late childhood and early adolescence, and is often associated with subsequent accrual of comorbid mental health problems such as major depression and substance use disorder. SAD is a multifactorial disease with both environmental and genetic factors as well as their interactions. Generalized SAD is a particularly severe form of SAD, involving all-encompassing fear of social situations. Social anxiety disorder performance only (SAD-PO) is a specifier of SAD based on DSM-5 criteria. This specific type of SAD is typically not associated with comorbid psychiatric conditions or innate behavioral inhibition, and usually has a later onset. SAD-PO arises in narrower circumstances, namely, when a sufferer must perform in public. PSA and symptoms thereof may arise in individuals meeting the diagnostic criteria for SAD, for SAD-PO, for both SAD and SAD-PO, or for neither SAD nor SAD-PO.

Symptoms of performance anxiety, PSA, SAD, and SAD-PO may include physiological biomarkers associated with acute stress response, such as elevated heart rate, blood pressure, body temperature, or increased sweating, and other physical, cognitive, and emotional symptoms, e.g., shaking or trembling, blushing, rapid or difficulty breathing, dizziness, nausea, restlessness, muscle tension, sleep disturbances, excessive rumination or worry, difficulty concentrating outside of what is causing the anxiety, a feeling of the sufferer's mind having gone blank, fear of judgment, fear of making a mistake, fear of embarrassment, fear of people looking at the sufferer, fear of certain situations, e.g., ordering at a restaurant or speaking in front of a crowd, engaging in negative self-talk, and other symptoms as known in the art. The emotional, cognitive, and physical symptoms often lead individuals afflicted with such anxiety to alter their behaviors, including refusing invitations, clinging to familiar people in group situations, and avoiding the situations, events, or circumstances that cause them anxiety. Symptoms may frequently manifest in a fear of public speaking, fear of performing, or fear of engaging in situations where the individual must be in front of others. Individuals suffering from acute anxiety including, e.g., performance anxiety, PSA, SAD, and SAD-PO may further be at risk of developing unhealthy coping mechanisms, including use of drugs and alcohol as a means to relax in anxiety-provoking situations.

Public speaking anxiety can be assessed using the Public Speaking Anxiety Scale (PSAS) is used to assess the three-component model of anxiety. The PSAS consists of seventeen items across three subscales: 1) cognitive (eight items), 2) behavioral (four items), and 3) physiological (five items). Each item has a score ranging from 1 (“not at all”) to 5 (“extremely”), with five items on the scale being reverse coded. The PSAS total score can range from 17 to 85, and is considered a highly reliable and comprehensive measure to assess public speaking anxiety. Current treatments for performance anxiety, SAD, and SAD-PO include psychological and pharmacological therapies, alone or in combination. As noted above, standard of care pharmacological interventions to address procedural anxiety invoke significant undesirable side effects and risks. The selective serotonin reuptake inhibitors (SSRIs) paroxetine, sertraline, and fluvoxamine, and the serotonin norepinephrine reuptake inhibitor (SNRI) venlafaxine have been approved by the FDA for SAD in the United States, and are usually the first choice of pharmacologic intervention for SAD. However, these drugs cannot be used for the treatment of acute anxiety due to slow onset of action. Additionally, the SSRIs increase synaptic serotonin levels, and approximately 40% of patients treated with SSRIs report sexual dysfunction associated with SSRI use. Other medications including monoamine oxidase inhibitors (MAOIs) and benzodiazepines (BZDs) have shown efficacy in placebo-controlled trials with small sample sizes. However, benzodiazepines are associated with adverse effects such as drowsiness, memory disturbances, sedation, and abuse liability. As a result, existing treatments are incompatible with, e.g. proficient performance in public speaking, musical performance, and other activities, and safety in the course of other activities, including, e.g., activities at heights. In addition, existing treatments offer high rates of only partial response, and low rates of long-term remission. Beta blockers such as propranolol have been used off-label to combat symptoms of performance anxiety, although insufficient evidence is available to support the routine use of propranolol in the treatment of acute anxiety.

Many patients with SAD, including SAD-PO, only experience significant distress in events that occur infrequently (e.g. public speaking or social gathering) or only benefit partially from psychological and pharmacological therapies. Therefore, there is an unmet medical need for more effective anxiolytics with improved safety profiles and that are non-sedating, including those that can be used on an as-needed basis, e.g., for the treatment of public speaking or performance anxiety in patients having or not having a diagnosis of SAD or SAD-PO.

The alpha-7 nicotinic acetylcholine receptor (α7-nAChR) is a homo-pentameric nAChR with both ionotropic and metabotropic functioning. The α7-nAChR is highly expressed in the brain and immune cells, including microglia. The α7-nAChR signaling pathway is involved in neurotransmitter release, cognitive functioning, and the cholinergic anti-inflammatory response. Genetic abnormalities such as copy number changes in the CHRNA7 gene that encodes the α7-nAChR have been implicated in several neurological and psychiatric disorders including e.g., schizophrenia and Alzheimer's disease (AD).

The compound (R)-3-(6-p-tolyl-pyridin-3-yloxy)-1-aza-bicyclo[2.2.2]octane, shown below:

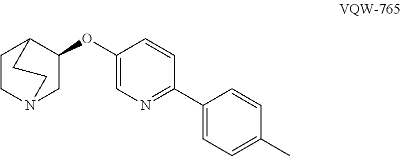 [SMILES: Cc1ccc(-c2ccc(O[C@H]3CN4CCC3CC4)cn2)cc1]

is a potent and selective α7 nAChR agonist disclosed in U.S. Pat. No. 7,579,362. The compound is also known as (R)-3-((6-(p-tolyl)pyridin-3-yl)oxy)quinuclidine, AQW-051, VQW-765, VQW, and by the IUPAC name, (3R)-3-{[6-(4-methylphenyl)pyridine-3-yl]oxy}-1-azabicyclo[2.2.2]octane. VQW-765 has a molecular weight of 294.39 g/mol, a pKa (predicted) of 9.09±0.33, and a LogP (predicted) of 3.447±0.402. Pharmaceutically acceptable acid addition salts of (R)-3-(6-p-Tolyl-pyridin-3-yloxy)-1-aza-bicyclo[2.2.2]octane, and particularly the mono-fumarate salt are disclosed in U.S. Pat. No. 9,365,565.

U.S. Pat. No. 7,579,362 describes VQW-765 as an α7 nAChR agonist useful for the prevention and treatment of psychotic disorders such as schizophrenia, mania, depression and anxiety, and for the prevention and treatment of neurodegenerative disorders such as senile dementia, Alzheimer's disease and other intellectual impairment disorders, such as attention deficit hyperactivity disorders (ADHD); Parkinson's disease, Huntington's chorea, amyotrophic lateral sclerosis, multiple sclerosis, epilepsy, convulsions, Tourette syndrome, OCD (obsessive compulsive disorder), neuropathic, postoperative and inflammatory pain, phantom limb pain, cognition, smoking cessation, memory deficits and dysfunction, learning deficit, panic disorders, narcolepsy, nociception, AIDS dementia, senile dementia, autism, tardive dyskinesia, social phobia, pseudodementia. International Patent Application Publication No. WO 2022/115526 A1, which is incorporated by reference herein, also discloses use of VQW-765 in the treatment of public speaking anxiety.

### BRIEF DESCRIPTION OF THE INVENTION

A first aspect of the disclosure provides a method for treating an individual suffering from or susceptible to acute anxiety, comprising: determining for said individual a dose of an α7 nAChR modulator providing an exposure level of the modulator known to be effective to ameliorate or prevent acute anxiety or a symptom thereof; and administering to the individual the α7 nAChR modulator at the dose found to provide the individual said exposure level.

A second aspect of the disclosure provides an improved method of treating an individual suffering from or susceptible to acute anxiety by administering to the individual an alpha 7 nicotinic acetylcholine receptor (α7 nAChR) modulator, at a dose effective to ameliorate or prevent the acute anxiety or a symptom thereof. According to this aspect, the improvement comprises selecting the individual for treatment with the α7 nAChR modulator based on whether or not the sex of the individual is female.

A third aspect of the disclosure provides a method of treating or preventing acute anxiety or a symptom thereof, in an individual in need of such treatment, the method comprising: administering to the individual an alpha 7 nicotinic acetylcholine receptor (α7 nAChR) modulator at a dose that is therapeutically effective to treat the acute anxiety or the symptom thereof.

A fourth aspect of the disclosure provides an improvement in a method of treatment of an individual suffering from acute anxiety or a symptom thereof, by administering to the individual an α7 nAChR modulator at a dose effective to treat the acute anxiety or the symptom thereof. In such method, the improvement comprises selecting the individual for treatment with the α7 nAChR modulator based on sex, wherein the sex of the individual is female.

A fifth aspect of the disclosure provides an improvement in a method of preventing a manifestation of one or more symptoms of acute anxiety in an individual by administering to said individual prior to experiencing an acute anxiety-inducing setting, an alpha 7 nicotinic acetylcholine receptor (α7 nAChR) modulator, at a dose effective to prevent said manifestation. In such method, the improvement comprises selecting the individual for treatment with the α7 nAChR modulator based on sex, wherein the sex of the individual is female.

A sixth aspect provides a method of treating an individual suffering from acute anxiety or a symptom thereof, comprising: administering to the individual an alpha 7 nicotinic acetylcholine receptor (α7 nAChR) modulator at an initial dose; and after an elapsed time period following administration of the initial dose, obtaining a biological sample from the individual. A concentration of the α7 nAChR modulator in the biological sample is measured; and prior to an anxiety-inducing setting or event, the method includes administering the α7 nAChR modulator to the individual at a therapeutic dose for the individual. If the concentration of the α7 nAChR modulator in the biological sample is below a first threshold level, the therapeutic dose is a first therapeutic dose that is larger than the initial dose. If the concentration of the α7 nAChR modulator in the biological sample is between the first threshold level and a second threshold level, the therapeutic dose is a second therapeutic dose that is equivalent to the initial dose. If the concentration of the α7 nAChR modulator in the biological sample is above the second threshold level, the therapeutic dose is a third therapeutic dose that is smaller than the initial dose.

A seventh aspect provides a method of determining a therapeutic or effective dose of an alpha 7 nicotinic acetylcholine receptor (α7 nAChR) modulator for treatment of an individual suffering from acute anxiety or a symptom thereof, according to methods described herein.

These and other aspects, advantages and salient features of the invention will become apparent from the following detailed description, which describes in greater details various embodiments of the invention.

### BRIEF DESCRIPTION OF THE DRAWINGS

FIG. 1 illustrates the Trier Social Stress Test (TSST) procedure discussed herein in Example 1.

FIGS. 2A, 2B, and 2C are graphs illustrating the SUDS rating across TSST phases for VQW-765 and placebo in the intent to treat (ITT) population, which includes male and female subjects (FIG. 2A), in female subjects only (FIG. 2B), and in male subjects only (FIG. 2C). The curves represent mean SUDS score (±SEM) in each timepoint of the TSST for groups treated with VQW-765 or placebo in the respective populations. In these graphs, baseline data are collected in a pre-dose phase; resting data are collected in the task introduction phase; A1-A3 data are collected in the anticipation phase; and P1-P5 data are collected in the performance phase. The (*) symbol indicates significant difference between VQW-765 and placebo with p-value<0.05.

FIG. 3 is a graph illustrating the VQW-765 exposure-response relationship in female study subjects.

FIG. 4 is a graph illustrating the efficacious exposure range for VQW-765 in female subjects, in which individual exposure is indicated by diamond shaped data points (♦) indicating VQW-765 concentration in plasma as measured after the TSST. VQW-L, VQW-M, and VQW-H indicate low, moderate, and high exposure, respectively.

FIGS. 5A, 5B, and 5C are graphs illustrating the relationship between plasma exposure of VQW-765 and response to treatment. The curves represent mean SUDS score (±SEM) at each timepoint of the TSST for the groups with different exposure of VQW-765 and placebo in the ITT population (FIG. 5A), female population (FIG. 5B) and male population (FIG. 5C). As described relative to FIGS. 2A-2C, baseline data are collected in a pre-dose phase; resting data are collected in the task introduction phase; A1-A3 data are collected in the anticipation phase; and P1-P5 data are collected in the performance phase. The (*) symbol indicates significant difference between subjects with efficacious exposure of VQW-765 and placebo with p-value<0.05.

FIGS. 6A, 6B, 6C, and 6D are graphs illustrating mean SUDS score (FIGS. 6A, 6B) and HRV change (FIGS. 6C, 6D) during the TSST for subjects with synchronized SUDS and HR data. FIGS. 6A and 6B illustrate mean SUDS scores (±SEM) in each timepoint of the TSST for groups treated with VQW-765 or placebo in the ITT and female populations, respectively. FIGS. 6C and 6D illustrate corresponding HRV changes during the TSST for the ITT and female populations, respectively. As above, baseline data are collected in a pre-dose phase; resting data are collected in the task introduction phase; A1-A3 data are collected in the anticipation phase; P1-P5 data are collected in the performance phase; pre-TSST data are collected post-dose but still in waiting room; and R1-R5 data are collected in the recovery phase after the public speaking challenge. The (*) symbol indicates significant difference between VQW-765 and placebo with p-value<0.05.

FIG. 7 is a graph illustrating the difference in mean SUDS rating across TSST phases in female subjects and male subjects in the placebo group. Curves represent mean SUDS score (±SEM) in each timepoint of the TSST for male and female subjects treated with placebo. As above, baseline data are collected in a pre-dose phase; resting data are collected in the task introduction phase; A1-A3 data are collected in the anticipation phase; and P1-P5 data are collected in the performance phase. The (*) symbol indicates significant difference between male and female with p-value<0.05.

FIG. 8 is a graph illustrating differences in heart rate between male and female subjects receiving placebo during the TSST. Curves represent mean heart rate (±SEM) in each timepoint of the TSST for male and female subjects treated with placebo. As above, baseline data are collected in a pre-dose phase; resting data are collected in the task introduction phase; A1-A3 data are collected in the anticipation phase; and P1-P5 data are collected in the performance phase.

FIG. 9 is a graph illustrating the SUDS pattern in subjects who did not wear a face mask during the TSST. Curves represent mean SUDS score (±SEM) in each timepoint of the TSST for groups treated with VQW-765 or placebo. As above, baseline data are collected in a pre-dose phase; resting data are collected in the task introduction phase; A1-A3 data are collected in the anticipation phase; and P1-P5 data are collected in the performance phase. The (*) symbol indicates significant difference between VQW-765 and placebo with p-value<0.05.

The figures are intended to depict only typical aspects of the disclosure, and therefore should not be considered as limiting the scope of the disclosure.

### DETAILED DESCRIPTION OF THE INVENTION

In various embodiments of the invention, methods are described herein for the treatment of an individual suffering from acute anxiety. As used herein, acute anxiety refers to individuals experiencing acute anxiety regardless of root cause and triggering event or setting, and without regard to whether the acute anxiety occurs within the context of social anxiety disorder (SAD), the performance only subtype of social anxiety disorder (SAD-PO), other diagnosis, or no diagnosis. Acute anxiety is understood to encompass performance anxiety, which may include public speaking anxiety (PSA), test taking anxiety, writer's block, musical performance anxiety, and other types of performance anxiety; procedural anxiety, for example medical or dental procedural anxiety; an acute phobia exposures, settings, and events such as, e.g., airplane boarding, take off, and landing, high height situations such as, e.g., mountain climbing and standing on an upper floor of a tall building, proximity to an anxiety-inducing animal, and other anxiety-inducing settings and/or phobic exposures.

References to an individual suffering from public speaking anxiety (PSA, also known as fear of public speaking, or fear of speaking in public) or performance anxiety may include any individual who experiences one or more symptoms of anxiety in association with public speaking or performance. Such anxiety may be referred to as “stage fright,” and may be brought on by public performance, for example, musical performance, athletic performance, acting performances, and public speaking, or anticipation of such public performance. Such individuals may meet many to all of the diagnostic criteria for SAD, including, e.g., marked anxiety or fear about performing in front of others (e.g., giving a speech). Individuals suffering from PSA may include individuals who are not diagnosed with SAD, but who nonetheless experience PSA. For example, under the DSM-5, an individual who is afraid to speak in public would not receive a diagnosis of social anxiety disorder if this activity is not routinely encountered on the job or in classroom work, and if the individual is not significantly distressed about it. Such individuals may nonetheless be considered to suffer from PSA, and benefit from treatment as described herein. Such individuals may further be identified through objective measures such as, e.g., the Public Speaking Anxiety Scale (PSAS), on which a score of 60 or greater is consistent with identification of an individual as having substantial public speaking anxiety.

As used herein, references to an individual suffering from social anxiety disorder may be considered to include individuals meeting diagnostic criteria for social anxiety disorder (SAD) as understood by those skilled in the art. For example, such individuals may meet the diagnostic criteria for social anxiety disorder set forth in the American Psychiatric Association's Diagnostic and Statistical Manual of Mental Disorders, 5th edition (DSM-5), pp. 202-208 (2013). Such references are considered to include both individuals diagnosed with SAD, and those who may meet the diagnostic criteria but have not been diagnosed for any reason including, e.g., not having presented for clinical care for SAD or symptoms thereof.

References to an individual suffering from social anxiety disorder-performance only (SAD-PO) may be considered to include individuals meeting diagnostic criteria for the SAD-PO subtype as understood by those skilled in the art. For example, such individuals may meet the diagnostic criteria for the social anxiety disorder-performance only specifier as set forth in the DSM-5. Such references are considered to include both individuals diagnosed with SAD-PO, and those who may meet the diagnostic criteria but have not been diagnosed for any reason including, e.g., not having presented for clinical care for SAD-PO or symptoms thereof.

As used herein, references to treatment of an individual with acute anxiety may be considered to include a reduction in severity of the individual's symptoms, prevention or attenuation of progression of acute anxiety in the individual, prevention or attenuation of the individual's symptoms in response to a situation or stimulus, or complete resolution of one or more of the individual's symptoms of acute anxiety, after such symptom or symptoms have manifested in the individual.

As used herein, references to an individual in need of such treatment refer to an individual who is either experiencing acute anxiety, or who desires or seeks prevention of a manifestation of acute anxiety due to an anticipated future triggering event. An individual in need of treatment may be in need of treatment or prevention of acute anxiety or symptoms thereof due to public speaking anxiety (PSA), performance anxiety, social anxiety disorder, social anxiety disorder-performance only (SAD-PO) subtype, and the like, as described herein.

In one embodiment, a method is provided for treating or preventing acute anxiety or a symptom thereof, in an individual in need of such treatment. Such method includes administering to the individual an alpha 7 nicotinic acetylcholine receptor (α7 nAChR) modulator at a dose that is therapeutically effective to treat the acute anxiety or the symptom thereof. Particularly, the α7 nAChR modulator may be an agonist or a partial agonist of the α7 nAChR, and may for example be selective for the α7 subtype of nAChR. More particularly, the α7 nAChR modulator may be (R)-3-(6-p-tolyl-pyridin-3-yloxy)-1-aza-bicyclo[2.2.2]octane or a pharmaceutically acceptable salt thereof. In certain embodiments, the individual may be female.

In certain embodiments, the acute anxiety to be treated or prevented may be caused by any of social anxiety disorder (SAD), SAD-performance only (SAD-PO), public speaking anxiety (PSA) independent of SAD diagnosis, public speaking anxiety as a presenting symptom of social anxiety disorder (SAD), public speaking anxiety as a presenting symptom of SAD-performance only (SAD-PO), performance anxiety, procedural anxiety, e.g., medical or dental, or exposure to an acute phobia setting or event.

In this and other methods described herein, the α7 nAChR modulator may be orally administered, e.g. in the form of a capsule or tablet containing the α7 nAChR modulator and at least one pharmaceutically acceptable excipient.

In this and other methods described herein for treating an individual with acute anxiety, a dose effective to produce the desired therapeutic response is an amount or dosage that is therapeutically effective, e.g. efficacious to treat the symptoms or underlying acute anxiety experienced by the individual, to shorten the course or lessen the severity of the manifestation of the condition or disorder, or to prevent, mitigate, or ameliorate symptoms of the condition or disorder. The dose for a human may be an amount between about 0.5 mg and about 700 mg as described in Table 1, for example, about 0.5 mg, about 1 mg, about 2 mg, about 2.5 mg, about 5 mg, about 7.5 mg, about 10 mg, about 15 mg, about 20 mg, about 25 mg, about 50 mg, about 75 mg, about 100 mg, about 200 mg, about 250 mg, about 500 mg, or about 700 mg.

TABLE 1
Exemplary doses

| Dose (mg/kg) | Dose for exemplary 70 kg human (mg) |
|---|---|
| 0.007 | 0.5 |
| 0.03 | 2 |
| 0.04 | 2.5 |
| 0.07 | 5 |
| 0.14 | 10 |
| 0.21 | 15 |
| 0.29 | 20 |
| 1.07 | 75 |
| 1.43 | 100 |
| 2.86 | 200 |
| 10 | 700 |

In certain embodiments described herein, the dose may particularly be about 5 mg, about 7.5 mg, about 10 mg, about 15 mg, or about 20 mg, or more particularly about 10 mg.

The dose of the active agent of the present invention may be varied so as to administer an amount of the active agent which is effective to provide the desired drug exposure level (as measured by, e.g., plasma concentration level) to a particular individual in order to achieve the desired therapeutic response for that particular individual without being toxic. The therapeutic dose will depend upon a variety of pharmacokinetic factors including the route of administration, the time of administration, the rate of excretion, the duration of the treatment, other drugs, compounds, or materials used in combination with the particular compositions employed, the age, sex, weight, condition, general health and prior medical history of the individual being treated, and like factors well known in the medical arts. In certain embodiments, the effective dose may be determined and/or confirmed to be a dose that causes a drug exposure level that falls within a range of exposure levels known to be efficacious. For example, the effective dose may be a dose that results in a concentration of VQW-765 in plasma of, e.g., about 1 pmol/mL to about 10 pmol/mL, or about 1.5 pmol/ml to about 8 pmol/ml, or about 1.57 pmol/mL to about 6.32 pmol/mL in a given individual at the relevant time. The dose given to an individual may be adjusted upward or downward for future use based on the exposure as quantified for a given individual following a particular dose. For example, if an individual experiences an exposure level of greater than, e.g., about 10 pmol/mL, about 8 pmol/mL following administration of 10 mg VQW-765, the effective dose may be reduced to less than 10 mg, e.g., to about 7.5 mg, about 5 mg, about 2.5 mg, about 2 mg, about 1 mg, or about 0.5 mg for such individual. Such reduction brings the resulting plasma concentration of VQW-765 post-administration into the exposure level associated with an effective dose. On the other hand, if an individual experiences an exposure level of less than about 1 pmol/mL or about 1.5 pmol/ml following administration of 10 mg VQW-765, the effective dose may be increased to greater than 10 mg, e.g., to about 15 mg, about 20 mg, about 25 mg, and so on for such individual, to bring the resulting plasma concentration of VQW-765 post-administration into the exposure level associated with an effective dose.

The compound of the present invention can be administered by one or more routes of administration using one or more of a variety of methods known in the art. Although oral administration is described herein, as will be appreciated by the skilled artisan, the route and mode of administration will vary depending upon the desired results. Routes of administration may include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, a composition can be administered by a non-parenteral route, such as a topical, epidermal or mucosal route of administration, for example, intranasally, orally, vaginally, rectally, sublingually or topically. The route of administration and the dose may be selected according to methods known and understood in the art, in order to provide the desired drug exposure level as measured, e.g., in drug concentration in plasma, and pharmacokinetic profile.

The active compounds can be prepared as a solid, immediate release form comprising the compound and one or more pharmaceutically acceptable excipients. Alternatively, the active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

Preferred therapeutic compositions are compositions for oral or transdermal administration. A composition for enteral or parenteral administration is, for example, a unit dosage form, such as a sugar-coated tablet, a tablet, a capsule, a suppository or an ampoule. The unit content of active ingredients in an individual dose need not in itself constitute a therapeutically effective amount, since such an amount can be reached by the administration of a plurality of dosage units. A composition according to the invention may contain, e.g., from about 10% to about 100%, preferably from about 20% to about 60%, of the active ingredients.

If not indicated otherwise, a pharmaceutical composition according to the invention is prepared in a manner known per se, e.g. by means of conventional mixing, granulating, sugar-coating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like. Because of their ease of administration, tablets and capsules represent the most advantageous oral dosage unit forms, in which case solid pharmaceutical carriers are obviously employed.

In particular, the compound may be orally administered in a solid immediate release form, e.g., a tablet or a capsule, comprising VQW-765 in an amount of, e.g., about 0.5 mg, about 1 mg, about 2 mg, about 2.5 mg, about 5 mg, about 7.5 mg, about 10 mg, about 15 mg, about 20 mg, about 25 mg, about 50 mg, about 75 mg, about 100 mg, about 200 mg, about 250 mg, about 500 mg, or about 700 mg of the compound, together with one or more pharmaceutically acceptable excipients.

Alternatively, the compound may be orally administered in a solid controlled release form comprising the compound and one or more pharmaceutically acceptable excipients. Controlled release forms may contain larger doses with lower bioavailability as compared to immediate release forms. In further embodiments, the compound may be orally administered in a liquid suspension form, or intravenously administered in a liquid suspension form in a dosage providing comparable exposure to the compound.

In another embodiment provided herein, an improved method is described for the treatment of an individual suffering from acute anxiety or from one or more symptoms thereof. In the improved method of this embodiment, an individual suffering from acute anxiety or a symptom thereof, may be treated by administering to the individual an alpha 7 nicotinic acetylcholine receptor (α7 nAChR) modulator at a dose effective to treat the acute anxiety or the symptom thereof. Particularly, the α7 nAChR modulator may be an agonist or a partial agonist of the α7 nACh receptor, and may for example be selective for the α7 subtype of nAChR. More particularly, the α7 nAChR modulator may be (R)-3-(6-p-tolyl-pyridin-3-yloxy)-1-aza-bicyclo[2.2.2]octane or a pharmaceutically acceptable salt thereof. In certain embodiments, the compound may be (R)-3-(6-p-tolyl-pyridin-3-yloxy)-1-aza-bicyclo[2.2.2]octane fumarate. In various embodiments, the therapeutic dose may be, e.g., about 5 mg, about 7.5 mg, about 10 mg, about 15 mg, or about 20 mg.

In such a method, the improvement comprises selecting the individual for treatment with the α7 nAChR modulator based on the sex of the individual. In particular embodiments, the sex of the individual selected for treatment may be female.

Such treatment of a selected individual with an α7 nAChR modulator may treat, prevent, or ameliorate symptoms of acute anxiety, caused by performance anxiety, public speaking anxiety, procedural anxiety, or an exposure to an acute phobia setting, event, or experience. Such anxiety may also be caused by social anxiety disorder (SAD), SAD-performance only (SAD-PO), public speaking anxiety (PSA) independent of SAD diagnosis, public speaking anxiety as a presenting symptom of social anxiety disorder (SAD), or public speaking anxiety as a presenting symptom of SAD-performance only (SAD-PO). Regardless of cause, symptoms of the acute anxiety may manifest physically, including, e.g., shaking, trembling, blushing, increased heart rate, increased blood pressure, increased body temperature, rapid breathing, difficulty breathing, dizziness, nausea, restlessness, increased sweating, muscle tension, and/or sleep disturbance. Symptoms may additionally or alternatively manifest cognitively or emotionally in the individual, including, e.g., excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and/or engaging in negative self-talk.

In another embodiment of a method of preventing a manifestation of one or more symptoms of acute anxiety in an individual, an improvement is provided herein. Said method may include, prior to an individual experiencing an anxiety-inducing setting or event, administering to the individual an alpha 7 nicotinic acetylcholine receptor (α7 nAChR) modulator, at a dose effective to prevent said manifestation. In this method, the improvement comprises selecting the individual for treatment with the α7 nAChR modulator based on sex, wherein the sex of the individual is female.

According to this embodiment, the α7 nAChR modulator may be an agonist or a partial agonist of the α7 nACh receptor, and may for example be selective for the α7 subtype of nAChR. More particularly, the α7 nAChR modulator may be (R)-3-(6-p-tolyl-pyridin-3-yloxy)-1-aza-bicyclo[2.2.2]octane or a pharmaceutically acceptable salt thereof. In certain embodiments, the compound may be (R)-3-(6-p-tolyl-pyridin-3-yloxy)-1-aza-bicyclo[2.2.2]octane fumarate. Further, the α7 nAChR modulator is in the form of a capsule containing the α7 nAChR modulator and at least one pharmaceutically acceptable excipient. The administering may particularly include orally administering the capsule. One or more orally administered capsules may provide the therapeutic dose to the individual, which may be, e.g., 5 mg, 7.5 mg, 10 mg, 15 mg, 20 mg, etc. as disclosed herein.

Such treatment of a selected individual with an α7 nAChR modulator may treat or ameliorate symptoms of acute anxiety, caused by performance anxiety, public speaking anxiety, procedural anxiety, or an exposure to an acute phobia setting, event, or experience. Such anxiety may also be caused by social anxiety disorder (SAD), SAD-performance only (SAD-PO), public speaking anxiety (PSA) independent of SAD diagnosis, public speaking anxiety as a presenting symptom of social anxiety disorder (SAD), or public speaking anxiety as a presenting symptom of SAD-performance only (SAD-PO). Regardless of cause, symptoms of the acute anxiety may manifest physically, including, e.g., shaking, trembling, blushing, increased heart rate, increased blood pressure, increased body temperature, rapid breathing, difficulty breathing, dizziness, nausea, restlessness, increased sweating, muscle tension, and/or sleep disturbance. Symptoms may additionally or alternatively manifest cognitively or emotionally in the individual, including, e.g., excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and/or engaging in negative self-talk.

Such treatment of a selected individual with an α7 nAChR modulator may prevent symptoms of acute anxiety, caused by performance anxiety, public speaking anxiety, procedural anxiety, or an exposure to an acute phobia setting, event, or experience. Such anxiety may also be caused by social anxiety disorder (SAD), SAD-performance only (SAD-PO), public speaking anxiety (PSA) independent of SAD diagnosis, public speaking anxiety as a presenting symptom of social anxiety disorder (SAD), or public speaking anxiety as a presenting symptom of SAD-performance only (SAD-PO). Regardless of cause, symptoms of the acute anxiety to be prevented may be those that manifest physically, including, e.g., shaking, trembling, blushing, increased heart rate, increased blood pressure, increased body temperature, rapid breathing, difficulty breathing, dizziness, nausea, restlessness, increased sweating, muscle tension, and/or sleep disturbance, and/or those that manifest cognitively or emotionally, including, e.g., excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and/or engaging in negative self-talk.

In another embodiment, a method is provided for treating acute anxiety, or at least one symptom thereof in an individual by administering to the individual an alpha 7 nicotinic acetylcholine receptor (α7 nAChR). Particularly, the α7 nAChR modulator may be an agonist or a partial agonist of the α7 nACh receptor, and may for example be selective for the α7 subtype of nAChR. More particularly, the α7 nAChR modulator may be (R)-3-(6-p-tolyl-pyridin-3-yloxy)-1-aza-bicyclo[2.2.2]octane or a pharmaceutically acceptable salt thereof. In certain embodiments, the compound may be (R)-3-(6-p-tolyl-pyridin-3-yloxy)-1-aza-bicyclo[2.2.2]octane fumarate. The compound may be administered at a dosage described herein as one that is therapeutically effective, i.e., a therapeutic dose. The method may include first administering to the individual the α7 nAChR modulator at an initial dose. In one example, in which the α7 nAChR modulator is (R)-3-(6-p-tolyl-pyridin-3-yloxy)-1-aza-bicyclo[2.2.2]octane, the initial dose may be, e.g., 10 mg, and may be given to the individual in advance, e.g., at least one day in advance, at least two days in advance, at least a week in advance, etc., of an event that is anticipated to produce anxiety (“an acute anxiety-inducing setting”). After an elapsed time period following administration of the initial dose of the α7 nAChR modulator, the method includes obtaining a biological sample from the individual. The biological sample may include, e.g., blood, plasma, serum, saliva, urine, et al. as is known in the art. In certain embodiments, the elapsed time period may be, e.g., about two (2) to about three (3) hours. The method further includes measuring a concentration of the α7 nAChR modulator in the biological sample. This concentration may then be used to determine the therapeutic dose to be given to the specific individual being treated for acute anxiety before or at the time of a future an acute anxiety-inducing setting or event. At such time, the method includes administering the α7 nAChR modulator to the individual at the therapeutic dose for the individual.

If the concentration of the α7 nAChR modulator in the biological sample is below a first threshold level, the therapeutic dose for the individual is a first therapeutic dose, that is larger than the initial dose. For example, if the concentration of the α7 nAChR modulator in the biological sample is below this first threshold level, the dose should be increased in the future from the initial dose in order to treat or prevent a future manifestation of acute anxiety. In certain embodiments, the first therapeutic dose is larger than the initial dose by a factor of two. For example, if the initial dose is 10 mg, then the first therapeutic dose may be 20 mg. In one embodiment, the first threshold level may be, e.g., about 400 to about 500 ng of α7 nAChR modulator compound per mL of plasma, such that the first therapeutic dose is administered in a case in which the individual's plasma concentration of VQW is less than about 400-500 ng/ml, or is about 410, about 420, about 430, about 440, about 450, about 460, about 470, about 480, about 490, or about 500 ng/mL. In another embodiment, the first threshold level may be, e.g., about 1 pmol or about 1.5 pmol of the α7 nAChR modulator compound (e.g., VQW-765) per mL of plasma, such that the first, larger therapeutic dose may be administered if the individual's plasma concentration of VQW-765 is less than about 1 pmol/mL or about 1.5 pmol/mL.

If the concentration of the α7 nAChR modulator in the biological sample is between the first threshold level and a second threshold level that is higher than the first threshold level, the therapeutic dose for the individual may be a second therapeutic dose. The second therapeutic dose may be smaller than the first therapeutic dose described above, and may also be equivalent to the initial dose, i.e., if the concentration of the α7 nAChR modulator in the biological sample is between the first and second threshold levels, the dose should neither be increased nor decreased from the initial dose in the future in order to treat or prevent a future manifestation of acute anxiety. In this instance, the second therapeutic dose may be administered in lieu of the first therapeutic dose described above. In certain embodiments, for example, the initial dose may be 10 mg, and the second therapeutic dose may also be 10 mg. The first threshold level may be, e.g., about 400-500 ng/ml of α7 nAChR modulator in plasma, or may be about 410, about 420, about 430, about 440, about 450, about 460, about 470, about 480, about 490, or about 500 ng/ml. In other embodiments, the first threshold level may be, e.g., about 1 pmol/mL or about 1.5 pmol/ml. The second threshold level may be e.g., about 1880 ng of α7 nAChR modulator per mL of plasma, or about 1800, about 1810, about 1820, about 1830, about 1840, about 1850, about 1860, about 1870, about 1880, about 1890, about 1900, about 1910, about 1920, about 1930, about 1940, about 1950, about 1960, about 1970, about 1980, about 1990, or about 2000 ng/mL of α7 nAChR modulator in plasma. In other embodiments, the second threshold level may be, e.g., about 8 pmol/mL or about 10 pmol/mL. Thus, the second therapeutic dose is administered in a case in which the individual's plasma concentration of α7 nAChR modulator is greater than about 400-500 ng/ml, and is less than 1800-2000 ng/mL, or greater than about 1.5 pmol/mL and less than about 8 pmol/mL, or greater than about 1 pmol/mL and less than about 10 pmol/mL.

If the concentration of α7 nAChR modulator in the biological sample is above the second threshold level, the therapeutic dose may be a third therapeutic dose. The third therapeutic dose may be smaller than the initial dose, i.e., if the concentration of the α7 nAChR modulator in the biological sample is above the second threshold level (as defined herein), the dose should be decreased from the initial dose to arrive at the third therapeutic dose in order to treat or prevent a future manifestation of acute anxiety. In this instance, the third therapeutic dose may be administered in lieu of either of the first and/or second therapeutic doses. The third therapeutic dose may be smaller than the initial dose by, e.g., a factor of one half. In certain embodiments, for example, the initial dose may be 10 mg, and the third therapeutic dose may be 5 mg. As described above, the second threshold level may be, e.g., about 1800-2000 ng of α7 nAChR modulator per mL of plasma, or about 8 or about 10 pmol/mL of plasma, such that the third therapeutic dose is administered in a case in which the individual's plasma concentration of VQW is greater than about 1800-2000 ng/mL or about 8 or about 10 pmol/mL.

As alluded above, a method of determining an effective or therapeutic dose of α7 nAChR modulator to treat an acute manifestation of anxiety is also provided herein. The foregoing steps of administering an initial dose, obtaining a biological sample, and measuring the α7 nAChR modulator concentration in the biological sample, may be performed in an individual who is anticipating a future anxiety-producing event, e.g., procedure, performance, or public speaking-based, in order to then perform a step of determining an effective therapeutic dose in advance of the acute need for treatment.

In another embodiment, an improvement is provided in a method of treating or preventing acute anxiety or a symptom thereof with an alpha 7 nicotinic acetylcholine receptor (α7 nAChR) modulator, in an individual in need of such treatment, and in whom a dose of the α7 nAChR modulator has been found to provide an exposure level of the α7 nAChR modulator to the individual known to be effective to treat or prevent the acute anxiety or the symptom thereof. According to this embodiment, the improvement comprises administering to the individual the α7 nAChR modulator at the dose found to provide to the individual the exposure level of the α7 nAChR modulator known to be effective to treat or prevent the acute anxiety or the symptom thereof. Such a dose may be found or known to provide to the individual the exposure level of the α7 nAChR modulator known to be effective to treat or prevent the acute anxiety or the symptom thereof, according to a method as described herein. As described herein, the exposure level may be, e.g., about 1.5 pmol/mL to about 8 pmol/mL, or about 1 pmol/mL to about 10 pmol/mL, and the dose may be, e.g., about 5 mg, about 7.5 mg, about 10 mg, about 15 mg, about 20 mg, etc. as disclosed herein.

In still another embodiment, an improvement in a method of treating an individual suffering from acute anxiety or a symptom thereof, or of preventing a manifestation of one or more symptoms of acute anxiety in the individual by administering to the individual an alpha 7 nicotinic acetylcholine receptor (α7 nAChR) modulator, at a dose effective to treat or prevent the acute anxiety or the symptom thereof. According to this embodiment, the improvement comprises selecting the individual for treatment with the α7 nAChR modulator based on the individual being of the female sex. The dose that is effective to treat or prevent the acute anxiety or the symptom thereof may be a dose that has previously been found or determined to provide to the particular individual in need of treatment, an exposure level of the α7 nAChR modulator that is known to be effective to treat or prevent the acute anxiety or the symptom thereof. As described herein, the exposure level may be, e.g., about 1.5 pmol/mL to about 8 pmol/mL, or about 1 pmol/mL to about 10 pmol/mL, and the dose may be, e.g., about 5 mg, about 7.5 mg, about 10 mg, about 15 mg, about 20 mg, etc. as disclosed herein.

In any of the foregoing methods, the α7 nAChR modulator compound may be (R)-3-(6-p-tolyl-pyridin-3-yloxy)-1-aza-bicyclo[2.2.2]octane either in free base form or in a pharmaceutically acceptable acid addition salt form. A pharmaceutically acceptable salt as used herein refers to a salt of a free form that is not toxic, biologically intolerable, or otherwise biologically undesirable. Preferred pharmaceutically acceptable salts are those that are pharmacologically effective and suitable for contact with the tissues of individuals without undue toxicity, irritation, or allergic response. Particularly preferred pharmaceutically acceptable acid addition salts of the present compound are described in U.S. Pat. No. 9,365,565, and include the fumarate, maleate, chloride, phosphate, succinate, or malonate acid addition salt form. In particular, the compound may be (R)-3-(6-p-tolyl-pyridin-3-yloxy)-1-aza-bicyclo[2.2.2]octane in mono-fumarate acid addition salt form, i.e. (R)-3-(6-p-tolyl-pyridin-3-yloxy)-1-aza-bicyclo[2.2.2]octane fumarate.

In another aspect, a compound is provided that is (R)-3-(6-p-tolyl-pyridin-3-yloxy)-1-aza-bicyclo[2.2.2]octane or a pharmaceutically acceptable salt thereof, for use in any of the methods of treatment described herein.

In another aspect, a pharmaceutical composition is provided, the pharmaceutical composition comprising a compound that is (R)-3-(6-p-tolyl-pyridin-3-yloxy)-1-aza-bicyclo[2.2.2]octane or a pharmaceutically acceptable salt thereof for use in any of the methods described herein.

In another aspect, a compound is provided that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in any of the methods described herein.

The skilled artisan will appreciate that additional embodiments may be selected by combining the embodiments above, or by reference to the examples given herein.

### EXAMPLE

A multicenter, randomized, double-blind, placebo-controlled study evaluates the efficacy and safety of VQW-765 in subjects with substantial public speaking anxiety.

The primary objectives are to assess the effect of a single oral dose of 10 mg VQW-765 relative to placebo on the anxiety rating as measured by the Subjective Units of Distress Scale (SUDS) during the performance phase of a TSST in the intent to treat (ITT) population and females, the Clinician Global Impression of Change (CGI-C) scale and Patient Global Impression of Change (PGI-C) scale in the ITT population after the TSST, and the exposure-response relationship. The secondary objectives are to assess the effect of a single oral dose of 10 mg VQW-765 relative to placebo on the SUDS rating during the performance phase of a TSST in subjects with baseline LSAS score≥60 and the safety and tolerability of VQW-765 during the study.

### Methods

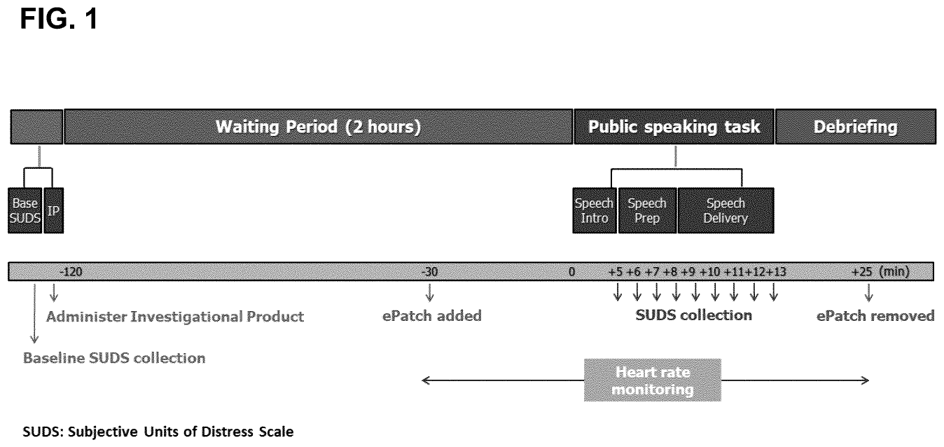

The study design is illustrated in FIG. 1 and Table 2 (below). Visit 1 includes screening for eligibility. At visit 2, subjects are asked for a baseline SUDS rating and then randomized to one of two arms in a 1:1 ratio. Subjects receive a single oral dose of 10 mg VQW-765 ((R)-3-(6-p-tolyl-pyridin-3-yloxy)-1-aza-bicyclo[2.2.2]octane fumarate) or placebo. VQW-765 is provided in the form of capsules that are white, opaque, hard gelatin, size 3, containing 10 mg VQW-765 and lactose monohydrate, microcrystalline cellulose, methocel, croscarmellose sodium, colloidal silicon dioxide, and magnesium stearate. Placebo capsules are provided in size and appearance identical to those containing VQW-765. Two hours later, subjects are instructed to prepare for a 5-minute speech for a mock job interview (resting phase). After 3 minutes of preparation (anticipation phase), the subject delivered the speech in front of an interview panel with two clinical staff (performance phase). SUDS rating was collected at each minute from the resting to performance phase.

TABLE 2
Clinical trial design

| | Visit 1 | Visit 2 |
|---|---|---|
| Phase | Screening for eligibility | Treatment (Day 1) |
| | (Day −14 to Day −1) | |
| Treatment | | VQW or placebo is given 2 |
| | | hours prior to a TSST |
| | Male and female | Randomization |
| | 18-70 years old | TSST |
| | PSAS ≥60 | SUDS, CGI-C, and PGI-C |
| | HAM-D score <18 | Biomarkers |
| | LSAS | PK samples |
| | Blood samples for | |
| | pharmacogenetics | |

During a screening phase (Table 2, visit 1), the Public Speaking Anxiety Scale (PSAS) is used to determine subjects with substantial public speaking anxiety, defined as having a PSAS score greater than or equal to 60. Clinical and laboratory assessments are also performed to assess each subject's eligibility. Inclusion criteria include a PSAS score greater than or equal to 60, non-smokers and non-nicotine users, and 17-item Hamilton Depression Rating Scale (HAM-D) score of less than or equal to 18. Exclusion criteria include history of bipolar disorder, schizophrenia, psychosis, seizures, OCD, PTSD, or substance or alcohol use disorder; concurrent psychotherapy; and psychotropic medication in the last six (6) months. Table 3 (below) provides demographics and key baseline measures for study subjects.

Eligible subjects are trained to use SUDS, a self-reported rating scale from 0 to 100, used to measure the intensity of anxiety during the TSST. A SUDS rating of “0” equals “no distress/totally relaxed” and 100 equals “highest anxiety/distress that you have ever felt.” In addition, the Liebowitz Social Anxiety Score (LSAS) is administered, and blood samples are collected for pharmacogenetic analysis. LSAS is a clinician-administered rating scale used to assess how social anxiety plays a role in daily life across a variety of situations. The LSAS is used to study outcomes in clinical trials, and includes 24 items to assess both social anxiety in situations and avoidance of those situations. Each item is rated as 0, none; 1, mild; 2, moderate; or 3, severe. The maximal score is 144.

TABLE 3
Demographics and key baseline measures

| | | | |
|---|---|---|---|
| All Randomized | VQW-765 | Placebo | Total |
| Subjects | (N = 116) | (N = 114) | (N = 230) |

Sex, n (%)

| | | | | | | |
|---|---|---|---|---|---|---|
| Female | 82 | (70.69) | 77 | (67.54) | 159 | (69.13) |
| Male | 34 | (29.31) | 37 | (32.46) | 71 | (30.87) |

Age (years)

| | | | | | | |
|---|---|---|---|---|---|---|
| Mean (SD) | 41.04 | (15.23) | 41.72 | (15.20) | 41.38 | (15.19) |

Race, n (%)

| | | | | | | |
|---|---|---|---|---|---|---|
| White | 66 | (56.90) | 59 | (51.75) | 125 | (54.35) |
| Black or African | 30 | (25.86) | 31 | (27.19) | 61 | (26.52) |
| American | | | | | | |
| Asian | 13 | (11.21) | 19 | (16.67) | 32 | (13.91) |
| American Indian or | 3 | (2.59) | 1 | (0.88) | 4 | (1.74) |
| Alaska Native | | | | | | |
| Native Hawaiian or | 1 | (0.86) | 1 | (0.88) | 2 | (0.87) |
| Other Pacific | | | | | | |
| Islander | | | | | | |
| Others | 3 | (2.59) | 3 | (2.63) | 6 | (2.61) |

Body Mass Index (kg/m²)

| | | | | | | |
|---|---|---|---|---|---|---|
| Mean (SD) | 28.10 | (6.24) | 29.10 | (7.32) | 28.60 | (6.80) |

17-item HAM-D

| | | | | | | |
|---|---|---|---|---|---|---|
| Mean (SD) | 4.18 | (4.06) | 4.32 | (4.28) | 4.25 | (4.16) |

PSAS

| | | | | | | |
|---|---|---|---|---|---|---|
| Mean (SD) | 69.99 | (6.44) | 70.39 | (6.79) | 70.19 | (6.60) |

LSAS

| | | | | | | |
|---|---|---|---|---|---|---|
| Mean (SD) | 83.14 | (24.65) | 83.50 | (26.31) | 83.32 | (25.43) |
| LSAS ≥60, n (%) | 97 | (83.62) | 92 | (80.70) | 189 | (82.17) |

After a waiting period of about two (2) hours from administration of study medication, subjects receive a modified Trier Social Stress Test (TSST) involving a public speaking challenge. The subject is instructed to prepare for a 5-minute speech for a job interview, and is asked for a SUDS rating (resting phase). Then the subject has 3 minutes to mentally prepare. A SUDS rating is collected at each minute interval (anticipation phase). Next, the subject gives a 5-minute speech to an audience of two clinical staff members dressed in white coats and previously unknown to the subject. A SUDS rating is collected just before the speech and then at each minute during the speech (performance phase). The speech is video recorded, and the video camera is observed by the subject. If the subject stops before 5 minutes and keeps silent for about 20 seconds, the lead clinical staff will say “you still have time.” If the subject does not continue, clinical staff will start asking questions until completion of performance phase. Physiological biomarkers associated with anxiety or acute stress response, including heart rate, blood pressure, body temperature, and sweating are monitored continuously by medical devices, including an ePatch Extended Holter Device throughout the Public Speaking challenge.

After the TSST debriefing, the biomarker measurement devices are removed, and Clinician Global Impression-Change (CGI-C) and Subject Global Impression-Change (PGI-C) are administered to assess the change relative to baseline on a 1 (very much improved) to 7 (very much worse) scale. Heart rate variability is calculated by the root mean square of successive differences between heart beats (RMSSD) method. Vital signs, safety labs, pharmacokinetic (PK) sample collection and electrocardiogram (ECG) are collected afterward.

A single blood sample is collected from all subjects after the TSST. Plasma is prepared within 60 minutes after blood collection and stored at −70° C. The plasma concentration of VQW-765 and its metabolites is determined by a LC-MS/MS method. Concentrations below 50 pg/mL are reported as below quantifiable limit (BQL).

### Results

Subjects: Of 459 subjects screened, 230 are enrolled and randomized to either VQW-765 (n=116) or placebo (n=114) treatment arms. All randomized subjects complete the study and are included in the analysis. At baseline, the mean measures of the 17-item HAM-D, PSAS, and LSAS are 4.3, 70.2, and 83.3, respectively (Table 3). There are no significant differences between the treatment groups on the demographic characteristics and the baseline measures. Of the 230 randomized subjects, 189 (82.2%) exhibit moderate to severe social anxiety symptoms as determined by the LSAS total score≥60 at baseline.

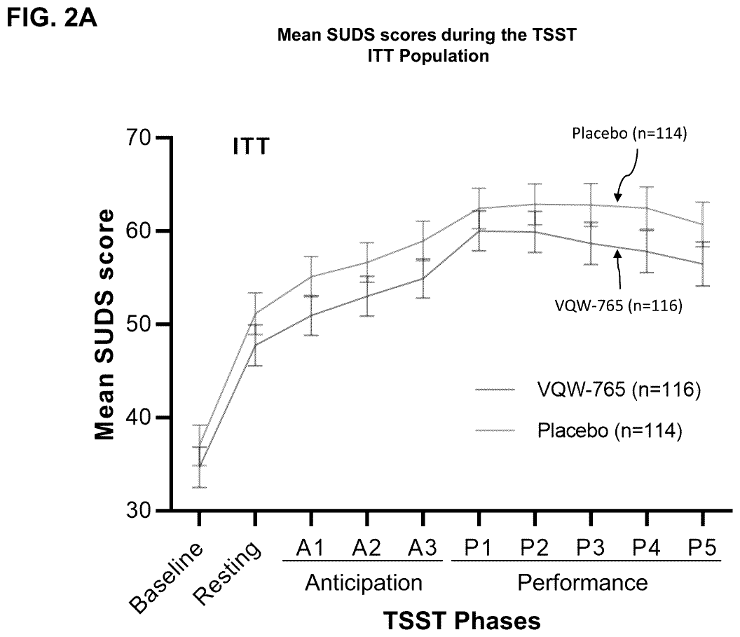

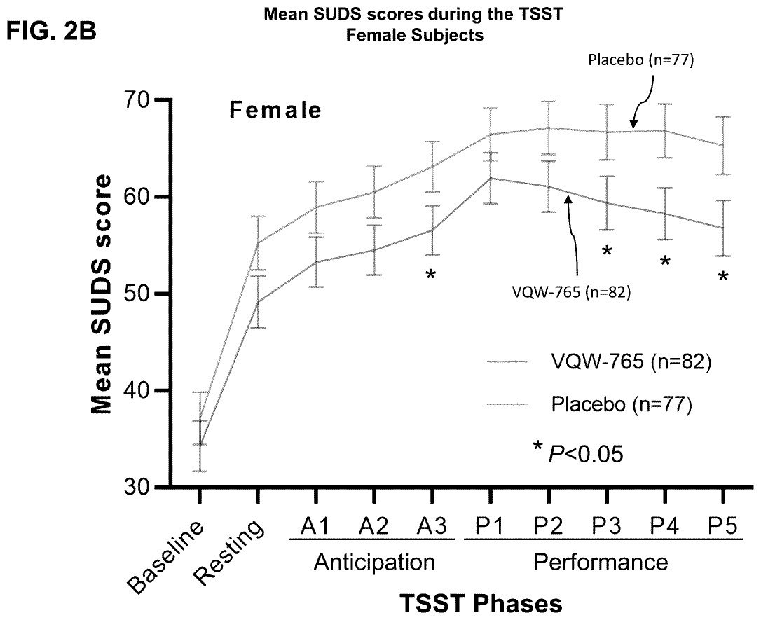

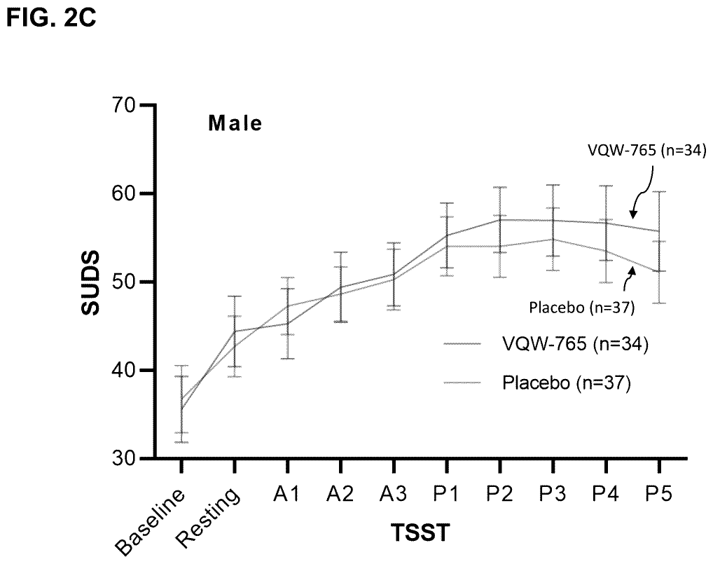

Primary Efficacy on SUDS: The principal assessment for the treatment effect is the self-reported anxiety rating as measured by the SUDS during the performance phase of a TSST. The SUDS rating is analyzed in the ITT population and females. Subjects receiving VQW-765 show a trend of improvement in intensity of anxiety as measured by the SUDS during the performance phase compared to placebo (p=0.116) (FIG. 2A, Table 4). Females (69.1% of the total subjects) receiving VQW-765 show a larger magnitude and significant response to VQW-765 (p=0.034) compared to placebo (FIG. 2B, Table 4). In contrast, males receiving VQW-765 do not show improvement on the SUDS rating compared to placebo (FIG. 2C).

TABLE 4
Primary and secondary efficacy analyses

| | | | VQW-765 | | | | |
|---|---|---|---|---|---|---|---|
| | | | (n = 116) | | Placebo (n = 114) | | |
| Population | Assessment | Phase | Mean | SE | Mean | SE | P-value |
| ITT | SUDS | Pre-dose | 34.66 | 2.16 | 37.02 | 2.18 | 0.2215 |
| | | Resting | 47.76 | 2.22 | 51.15 | 2.25 | 0.1423 |
| | | Anticipation | 52.96 | 2.05 | 56.90 | 2.08 | 0.0890 |
| | | Performance | 58.60 | 2.15 | 62.26 | 2.17 | 0.1164 |
| | CGI-C | Post-TSST | 3.6 | 0.11 | 3.6 | 0.11 | 0.4038 |
| | PGI-C | Post-TSST | 3.4 | 0.12 | 3.5 | 0.12 | 0.3993 |
| | | | VQW-765 (n = 82) | | Placebo (n = 77) | | P-value |
| Female | SUDS | Pre-dose | 34.27 | 2.63 | 37.14 | 2.71 | 0.2239 |
| | | Resting | 49.15 | 2.68 | 55.26 | 2.78 | 0.0576 |
| | | Anticipation | 54.80 | 2.47 | 60.88 | 2.56 | 0.0446 |
| | | Performance | 59.55 | 2.60 | 66.42 | 2.69 | 0.0342 |
| | | | VQW-765 (n = 56) | | Placebo (n = 114) | | P-value |
| ITT- | SUDS | Pre-dose | 35.89 | 3.13 | 37.02 | 2.20 | 0.3845 |
| 50% | | Resting | 46.25 | 3.20 | 51.15 | 2.25 | 0.1059 |
| quartile | | Anticipation | 50.89 | 2.92 | 56.90 | 2.06 | 0.0472 |
| | | Performance | 55.36 | 3.07 | 62.26 | 2.15 | 0.0332 |
| | | | VOW-765 (n = 38) | | Placebo (n = 77) | | P-value |
| Female- | SUDS | Pre-dose | 36.58 | 3.90 | 37.14 | 2.74 | 0.4530 |
| 50% | | Resting | 47.63 | 3.94 | 55.26 | 2.78 | 0.0577 |
| quartile | | Anticipation | 53.07 | 3.58 | 60.88 | 2.53 | 0.0384 |
| | | Performance | 54.37 | 3.74 | 66.42 | 2.62 | 0.0046 |
| | | | VQW-765 (n = 97) | | Placebo (n = 92) | | P-value |
| ITT- | SUDS | Pre-dose | 37.01 | 2.34 | 39.78 | 2.50 | 0.3225 |
| LSAS ≥ 60 | | Resting | 49.28 | 2.40 | 52.97 | 2.59 | 0.1509 |
| | | Anticipation | 54.05 | 2.34 | 57.62 | 2.33 | 0.1535 |
| | | Performance | 59.67 | 2.51 | 62.85 | 2.38 | 0.1664 |

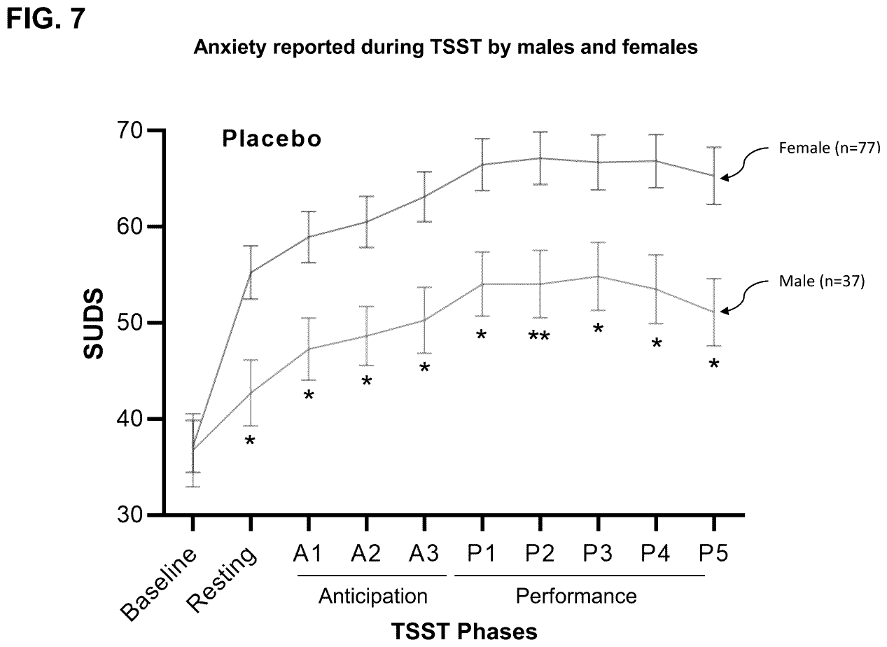

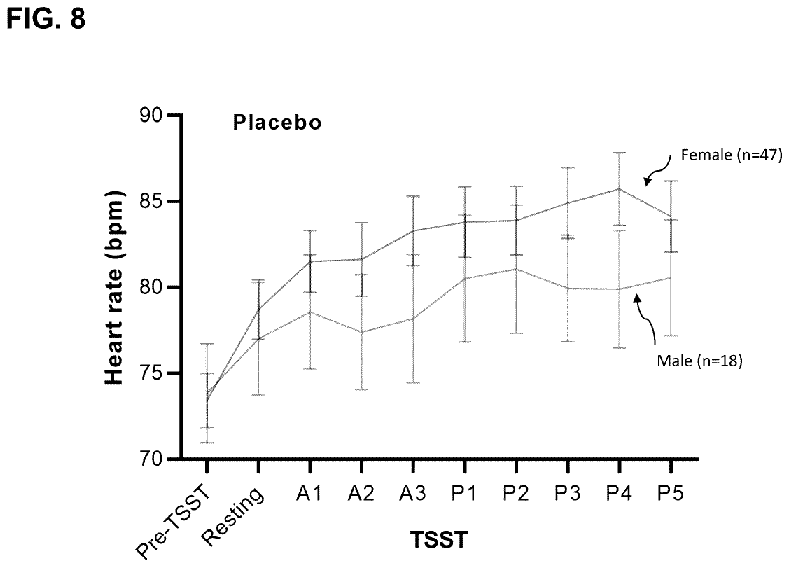

Females report much higher anxiety than males during the TSST when treated with placebo (FIG. 7), and demonstrate greater increase in heart rate than males during the TSST (FIG. 8). The differences in the SUDS rating between males and females are significant from the task introduction (resting phase) to the end of the speech. The results may suggest that lack of response in males could be due, at least in part, to the sex difference in response to the TSST.

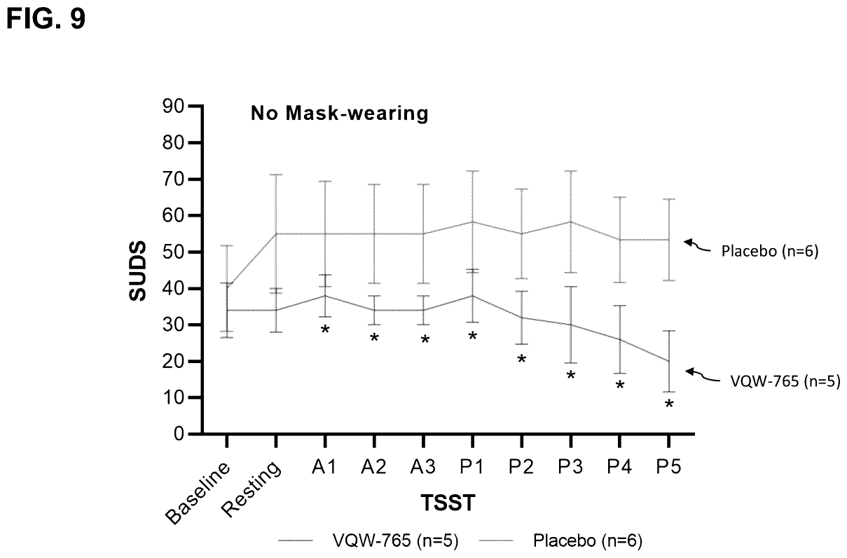

In addition, of the 230 subjects enrolled, 219 (95%) wear face masks during the clinical visits including the TSST procedure. It is possible that mask-wearing reduces the stress reactivity during the TSST and consequently reduces the treatment effect of VQW-765 over placebo. A post-hoc analysis assesses the treatment effect in subjects not wearing a face mask during the TSST (n=5 on VQW-765 and n=6 on placebo). As shown in FIG. 9, unmasked subjects receiving VQW-765 are significantly less anxious than those on placebo during the TSST, and the effect size is large.

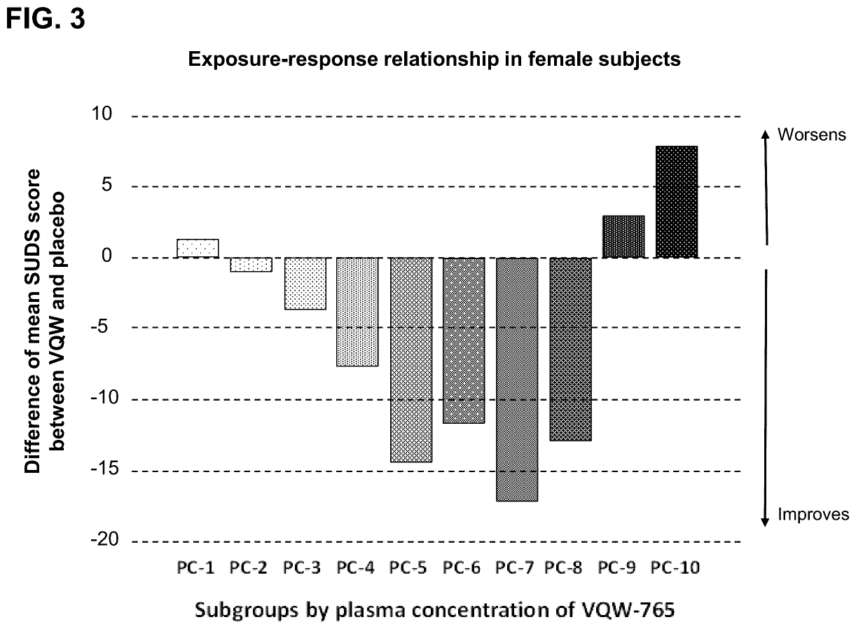

Exposure and Response Relationship: Exposure to VQW-765, i.e., amount of drug measured in blood plasma, is significantly related to clinical response to treatment. As shown in FIG. 3, VQW-765 demonstrates a U-shaped exposure-response relationship in female subjects. Mean plasma concentrations of VQW-765 for each subgroup shown along the X axis of the graph in FIG. 3 (PC-1, PC-2, PC-3, PC-4, PC-5, PC-6, PC-7, PC-8, PC-9, and PC-10) are provided below in Table 5.

TABLE 5
Mean plasma concentrations by exposure subgroup

| Exposure Group | Mean plasma concentration (pmol/ml) |
|---|---|
| PC-1 (n = 7) | 0.25 |
| PC-2 (n = 7) | 1.18 |
| PC-3 (n = 8) | 1.58 |
| PC-4 (n = 8) | 2.06 |
| PC-5 (n = 8) | 2.9 |
| PC-6 (n = 8) | 3.82 |
| PC-7 (n = 8) | 5.1 |
| PC-8 (n = 8) | 7.04 |
| PC-9 (n = 8) | 9.79 |
| PC-10 (n = 7) | 13.39 |
| Pbo (n = 77) | 0 |

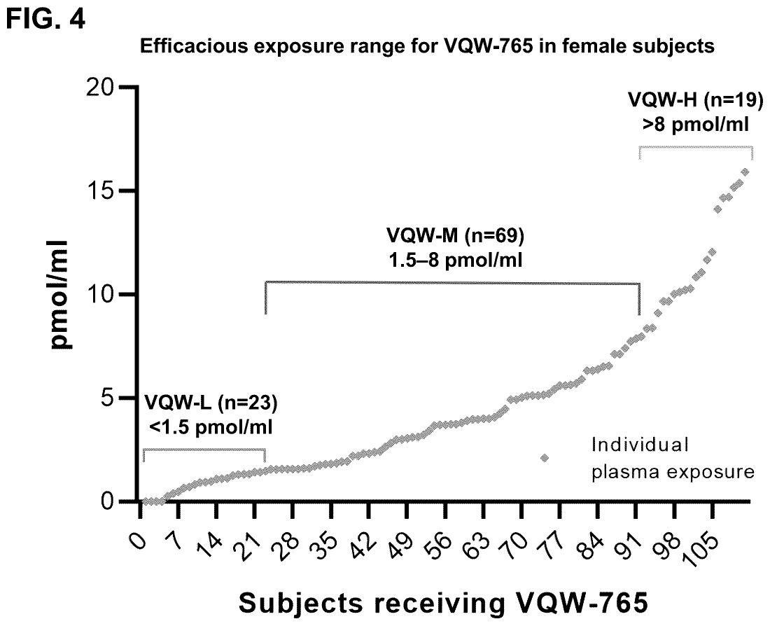

Subjects in the middle 50%, i.e., second and third quartiles (identified in FIG. 4 as “VQW-M” (n=69) (1.5-8 pmol/ml)), show significant improvement on the SUDS rating compared to placebo (p=0.033). This includes exposure groups PC-3, PC-4, PC-5, PC-6, PC-7, and PC-8 in FIG. 3 and Table 5.

In contrast, the subjects in the first quartile (identified in FIG. 4 as “VQW-L” (n=23) (<1.5 pmol/ml)) and fourth quartile (identified in FIG. 4 as “VQW-H” (n=19) (>8 pmol/ml) show similar performance on the SUDS rating as placebo (Table 4). First quartile exposure groups include PC-1 and PC-2 (FIG. 3 and Table 5), while fourth quartile exposure groups include PC-9 and PC-10 (FIG. 3 and Table 5).

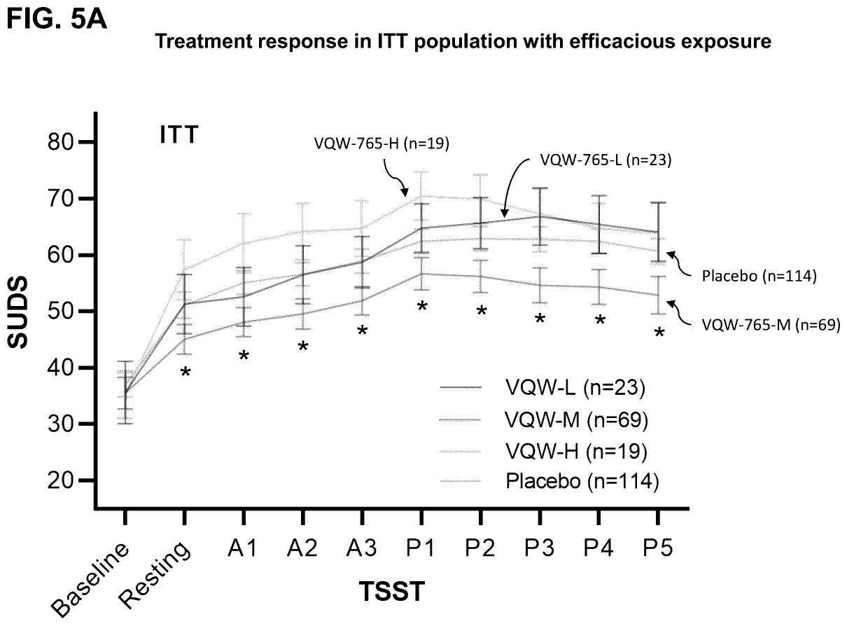

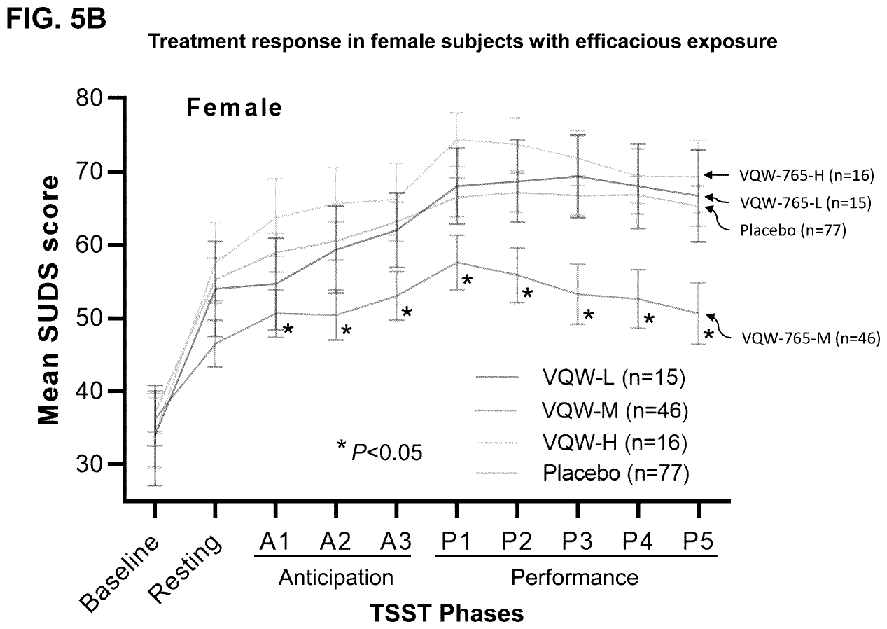

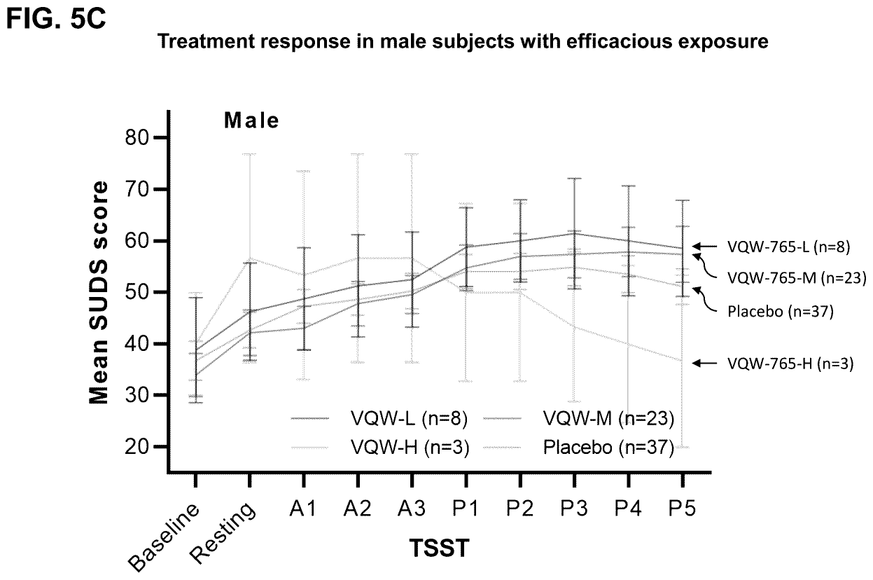

The VQW-765 plasma concentrations in the middle 50%, i.e., second and third quartiles, range from 1.57-6.32 pmol/mL. Based on the corresponding SUDS rating, efficacious exposure is observed over a range including about 1.5 pmol/mL to about 8.0 pmol/mL (FIG. 4). A post-hoc analysis shows that performance anxiety is significantly alleviated in subjects with efficacious exposures of VQW-765 compared to placebo (p=0.020) (FIG. 5A, Table 6). The treatment response is driven by females (p=0.002) (FIG. 5B, Table 6). Males with efficacious exposures of VQW-765 do not show improvement compared to placebo (FIG. 5C).

TABLE 6
Post-hoc efficacy analyses

| | | | | | Placebo | | |
|---|---|---|---|---|---|---|---|
| | | | VQW-765 (n = 69) | | (n = 114) | | P- |
| Population | Assessment | Phase | Mean | SE | Mean | SE | value |
| ITT- | SUDS | Pre-dose | 35.51 | 2.83 | 37.02 | 2.19 | 0.3368 |
| Efficacious | | Resting | 45.07 | 2.61 | 51.15 | 2.33 | 0.0471 |
| exposure | | Anticipation | 49.86 | 2.50 | 56.90 | 2.06 | 0.0165 |
| | | Performance | 54.96 | 2.94 | 62.26 | 2.10 | 0.0202 |
| | CGI-C | Post-TSST | 3.5 | 0.14 | 3.6 | 0.10 | 0.4343 |
| | PGI-C | Post-TSST | 3.4 | 0.14 | 3.5 | 0.10 | 0.5525 |
| | | | | | | | P- |
| | | | VQW-765 (n = 46) | | Placebo (n = 77) | | value |
| Female- | SUDS | Pre-dose | 36.30 | 3.72 | 37.14 | 2.70 | 0.4267 |
| Efficacious | | Resting | 46.52 | 3.23 | 55.26 | 2.93 | 0.0282 |
| exposure | | Anticipation | 51.38 | 3.20 | 60.88 | 2.56 | 0.0116 |
| | | Performance | 54.00 | 3.80 | 66.42 | 2.55 | 0.0029 |
| | CGI-C | Post-TSST | 3.4 | 0.19 | 3.7 | 0.13 | 0.2417 |
| | PGI-C | Post-TSST | 3.4 | 0.18 | 3.5 | 0.13 | 0.4655 |

Other Primary Efficacy Measures: No significant differences between VQW-765 and placebo are observed on either PGI-C (p=0.399) or CGI-C (p=0.404) (Table 4). The post-hoc analysis for subjects with efficacious exposures of VQW-765 also does not reveal any significant difference between VQW-765 and placebo on either PGI-C (p=0.376) or CGI-C (p=0.325) (Table 6).

Secondary Efficacy Measure: A formal diagnosis for SAD is not required for study participation, and only a few subjects have a history of SAD. Most subjects (82.2%) exhibit moderate to severe social anxiety symptoms as measured by the LSAS total score≥60 at baseline. An efficacy analysis is performed for those with moderate to severe social anxiety symptoms (see Table 6). As summarized in Table 6, the response to VQW-765 in the subset of subjects exhibiting moderate to severe social anxiety symptoms is similar to the overall study population. Performance anxiety is significantly reduced in those with efficacious exposures of VQW-765 (p=0.014), especially for females (p=0.004), as compared to placebo.

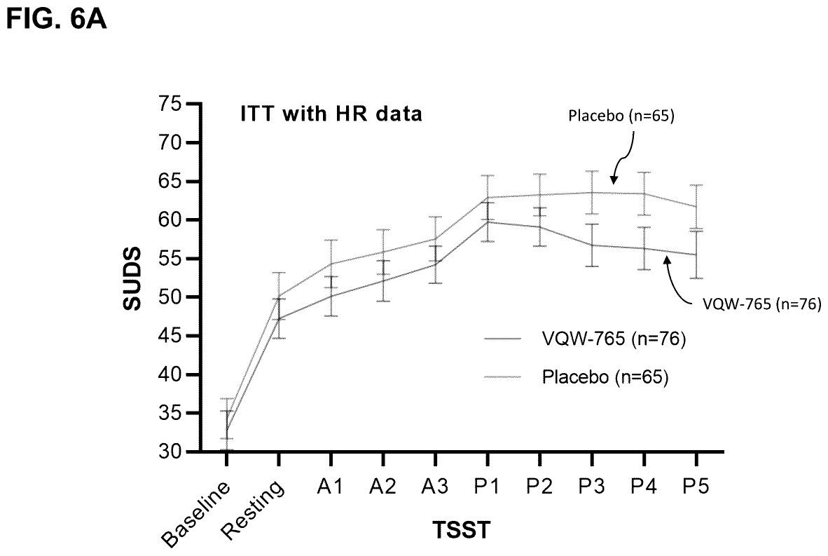

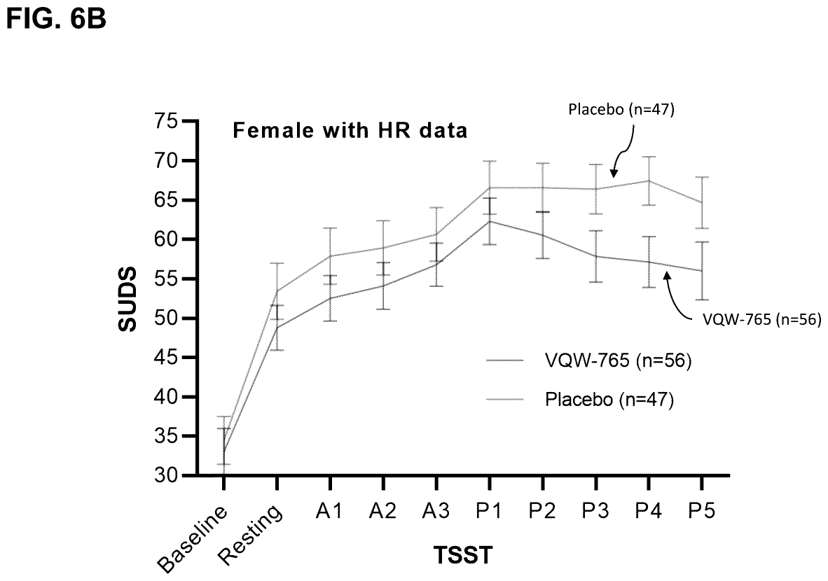

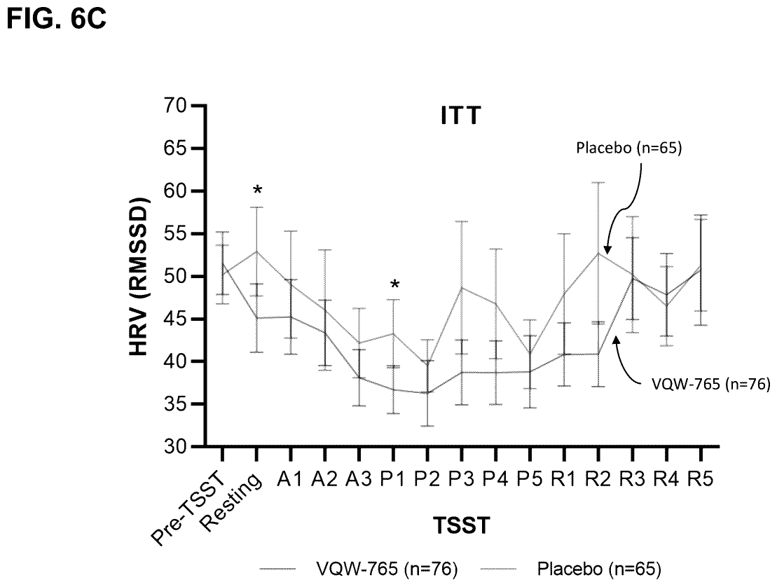

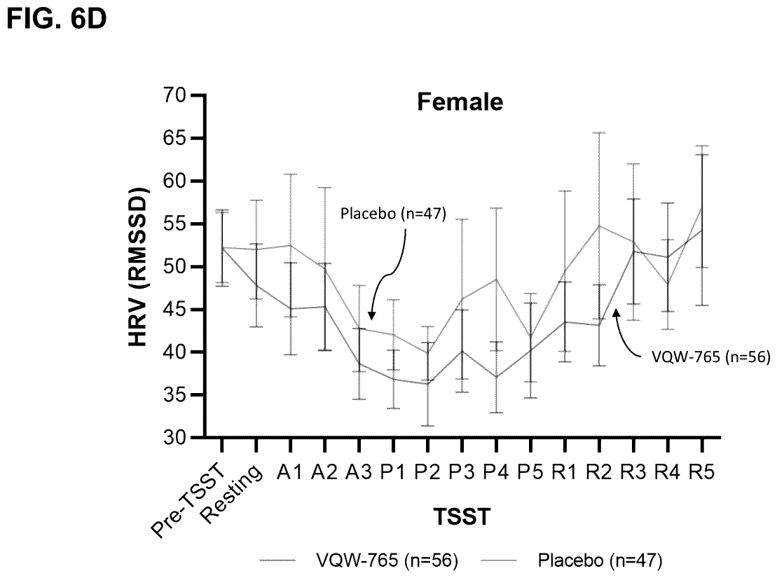

Heart rate variability (HRV): Of the 230 subjects enrolled, 141 have synchronized heart rate (HR) and SUDS rating data, and are included in the analysis. HRV is calculated by RMSSD, that is the most used time-domain method and reflects the beat-to-beat variance in HR. The pattern of the SUDS rating in those with HR data (FIGS. 6A, 6B) is similar to the overall study population. There are no significant differences in the SUDS rating and HRV between the treatment groups before the TSST (FIGS. 6C, 6D, “Pre-TSST”). When subjects enter the TSST room and are told they have 3 minutes to prepare for a 5-minute speech (resting phase), the SUDS rating immediately increases regardless of treatment (FIGS. 6A, 6B). However, the pattern of HRV change at this point is different between the treatment groups, with a significant decline in subjects receiving VQW-765 but a slight increase or no change in those on placebo (FIGS. 6C, 6D). The HRV starts to recover during the performance and recovery phases in both treatment groups, but the pattern of HRV increase is steady in subjects receiving VQW-765 and fluctuates in those on placebo (FIGS. 6C, 6D).

### Conclusions

An inverted U-shaped exposure-response curve is observed in humans, with a VQW-765 plasma concentration window of about 1.5 pmol/mL to about 8 pmol/mL for efficacious treatment of performance anxiety. Subjects, especially females, with efficacious exposures of VQW-765, i.e. exposures within the foregoing window, demonstrate significant and clinically meaningful improvement in the intensity of anxiety during a TSST involving a public speaking challenge. VQW-765 shows a similar effect during the anticipation phase of a TSST in those with efficacious exposures of VQW-765 (p-0.016), especially for females (p=0.011). No observed negative cognitive effects are reported by any subject, and VQW-765 is safe and well tolerated.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed, but that the invention will include all embodiments falling within the scope of the appended claims.

What is claimed is:

1. A method for treating an individual suffering from or susceptible to acute anxiety, comprising:
determining for said individual a dose of an α7 nAChR modulator providing an exposure level of the modulator known to be effective to ameliorate or prevent acute anxiety or a symptom thereof; and
administering to the individual the α7 nAChR modulator at the dose found to provide the individual said exposure level.

2. The improvement of claim 1, wherein the α7 nAChR modulator is an agonist or a partial agonist of the α7 nAChR.

3. The method of claim 1, wherein the α7 nAChR modulator is (R)-3-(6-p-tolyl-pyridin-3-yloxy)-1-aza-bicyclo[2.2.2]octane or a pharmaceutically acceptable salt thereof.

4. The method of claim 3, wherein the exposure level of the α7 nAChR modulator known to be effective to ameliorate or prevent acute anxiety or the symptom thereof is from about 1.5 pmol/mL to about 8 pmol/mL.

5. The method of claim 3, wherein the dose is about 5 mg.

6. The method of claim 3, wherein the dose is about 10 mg.

7. The method of claim 3, wherein the dose is about 20 mg.

8. The method of claim 1, wherein the α7 nAChR modulator is in the form of a capsule containing the α7 nAChR modulator and at least one pharmaceutically acceptable excipient, and wherein the administering further comprises orally administering the capsule.

9. The method of claim 1, wherein a cause of the acute anxiety is selected from the group consisting of: social anxiety disorder (SAD), SAD-performance only (SAD-PO), public speaking anxiety (PSA) independent of SAD diagnosis, public speaking anxiety as a presenting symptom of social anxiety disorder (SAD), public speaking anxiety as a presenting symptom of SAD-performance only (SAD-PO), performance anxiety, procedural anxiety, and an acute phobia.

10. The method of claim 1, wherein the individual is female.

11. In a method of treating an individual suffering from or susceptible to acute anxiety, by administering to the individual an alpha 7 nicotinic acetylcholine receptor (α7 nAChR) modulator, at a dose effective to ameliorate or prevent the acute anxiety or a symptom thereof, the improvement comprising:
selecting the individual for treatment with the α7 nAChR modulator based on whether or not the sex of the individual is female.

12. The improvement of claim 11, wherein the method comprises preventing the manifestation of one or more symptoms of acute anxiety in the individual, and wherein the administering occurs prior to the individual experiencing an acute anxiety-inducing setting.

13. The improvement of claim 12, wherein the administering occurs about two (2) hours prior to the individual experiencing an acute anxiety-inducing setting.

14. The improvement of claim 11, wherein a cause of the acute anxiety is selected from the group consisting of: social anxiety disorder (SAD), SAD-performance only (SAD-PO), public speaking anxiety (PSA) independent of SAD diagnosis, public speaking anxiety as a presenting symptom of social anxiety disorder (SAD), public speaking anxiety as a presenting symptom of SAD-performance only (SAD-PO), performance anxiety, procedural anxiety, and an acute phobia.

15. The improvement of claim 11, wherein the α7 nAChR modulator is an agonist or a partial agonist of the α7 nAChR.

16. The improvement of claim 11, wherein the α7 nAChR modulator is (R)-3-(6-p-tolyl-pyridin-3-yloxy)-1-aza-bicyclo[2.2.2]octane or a pharmaceutically acceptable salt thereof.

17. The improvement of claim 16, wherein the dose is about 5 mg, about 10 mg, or about 20 mg.

18. The improvement of claim 11, wherein the α7 nAChR modulator is in the form of a capsule containing the α7 nAChR modulator and at least one pharmaceutically acceptable excipient, and wherein the administering further comprises orally administering the capsule.

19. The improvement of claim 11, wherein the symptom is selected from the group consisting of: shaking, trembling, blushing, increased heart rate, increased blood pressure, increased body temperature, rapid breathing, difficulty breathing, dizziness, nausea, restlessness, increased sweating, muscle tension, and sleep disturbance,
or from the group consisting of: excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.

20. The improvement of claim 11, wherein the dose of the α7 nAChR modulator is known to provide an exposure level of the α7 nAChR modulator to the individual in need of treatment that is associated with efficacious treatment or prevention of the acute anxiety or the symptom thereof.
